(19) United States
(12) Patent Application Publication
(10) Pub. No.: US 2020/0114086 A1
(43) Pub. Date: Apr. 16, 2020

(54) SPEED CONTROLLED EMBALMING SYSTEM AND METHOD OF USING THE SAME

(71) Applicant: Joseph Mario Bernaudo, Bay Shore, NY (US)

(72) Inventor: Joseph Mario Bernaudo, Bay Shore, NY (US)

(21) Appl. No.: 16/592,929

(22) Filed: Oct. 4, 2019

Related U.S. Application Data
(60) Provisional application No. 62/745,974, filed on Oct. 15, 2018.

(51) Int. Cl. A61M 5/315; G01L 19/00; A01N 1/02

(52) U.S. Cl. CPC A61M 5/31515; A61M 2005/3128; A01N 1/02; G01L 19/003

(57) ABSTRACT

A portable machine for arterial embalming includes a tank assembly and a control assembly. The control assembly includes a fluid pressure gauge fluidly connected to a fluid output of a syringe vessel assembly of the tank assembly; a fluid injection tube fluidly connected to the fluid pressure gauge and configured for injection into the human cadaver body; a motor connected to a push rod that is connected to the syringe vessel assembly, wherein the motor drives the push rod to actuate the syringe vessel assembly to produce a flow of fluid from the tank assembly to the fluid pressure gauge; and a motor speed controller connected for varying a speed of the motor and for varying a maximum fluid pressure measured by the fluid pressure gauge, and, in turn, pressure filtration of fluid entering the human cadaver body.

### CROSS-REFERENCE TO RELATED APPLICATION

The present application claims the benefit of and priority to U.S. Provisional Patent Application Ser. No. 62/745,974, filed on Oct. 15, 2018, the entire content of which is incorporated by reference herein.

### BACKGROUND

### Technical Field

The present disclosure relates to a device used for fluid displacement and measurement of fluid resistance during arterial embalming. More specifically, the present disclosure relates to a speed controlled syringe pump embalming system and method of using the speed controlled syringe pump embalming machine.

### Description of Related Art

In order to restore and preserve the human body for anatomical or funeral purposes embalming fluid is forced into the vascular system removing and replacing the blood. Today, there are several embalming machines which regulate pressure and flow from the machine. However, regulation of the embalming machine pressure and flow does not provide an indication of the pressure or flow within the body.

The system of the present disclosure allows the embalmer to quantifiably measure the resistance of fluid from point of injection as it passes from the arterial system into the tissues of the human body and thus returning via the venous system.

Present embalming machines contain motor driven pumps and control the path of fluid travel from the machine as well as back to the fluid tank by way of plumbing valves. Thus measurements of pressure are not a true representation of fluid force being applied to the vascular system.

Instead of this method, the system of the present disclosure pumps fluid much like the human heart. The embalmer may easily control the speed at which fluid will be pumped and each pump will displace a predetermined volume of fluid so as to emulate the pulse of the human heart.

Because one licensed embalmer may be employed at several establishments, the system of the present disclosure is easily portable aiding the transport of equipment from one location to another.

### SUMMARY

According to an aspect of the present disclosure, a portable machine for arterial embalming is provided. The embalming machine includes a tank assembly and a control assembly associated with the tank assembly.

The tank assembly includes a fluid tank defining a reservoir of fluid for embalming a human cadaver body; and a syringe vessel assembly supported on the fluid tank and extending into the reservoir of the fluid tank, wherein the syringe vessel assembly is a one-way fluid valve, and wherein the syringe vessel assembly includes a fluid input and a fluid output.

The control assembly includes a fluid pressure gauge fluidly connected to the fluid output of the syringe vessel assembly; a fluid injection tube having a proximal end fluidly connected to the fluid pressure gauge and a distal end configured for injection into the human cadaver body; a motor connected to a proximal end of a push rod, a distal end of the push rob being connected to the syringe vessel assembly, wherein activation of the motor drives the push rod to actuate the syringe vessel assembly to produce a one-way flow of fluid from the reservoir of the fluid tank to the fluid pressure gauge and to the fluid injection tube; and a motor speed controller connected to the motor, wherein actuation of the motor speed controller varies a speed of the motor and varies a maximum fluid pressure measured by the fluid pressure gauge.

The fluid pressure gauge measures pressure filtration of fluid entering the human cadaver body.

The syringe vessel assembly may include an outer tubular sleeve having a distal end disposed within the reservoir of the fluid tank; a tubular syringe piston translatably disposed within the outer tubular sleeve, wherein a proximal end of the tubular syringe piston defines the fluid output and is fluidly connected to the fluid pressure gauge; and a seal interposed between an outer surface of the tubular syringe piston and an inner surface of the outer tubular sleeve.

The outer tubular sleeve of the syringe vessel assembly may include an opening formed in a distal end thereof, wherein the distal end of the outer tubular sleeve is in close proximity to a bottom of the fluid tank.

The tubular syringe piston of the syringe vessel assembly may include an opening formed in a distal end thereof, and wherein the syringe vessel assembly includes a ball disposed within the tubular syringe piston, wherein the ball has a diameter that is larger than a diameter of the opening formed in the distal end of the tubular syringe piston.

The push rod of the control assembly may be connected to the tubular syringe piston.

The control assembly may include a crank shaft having a proximal end non-rotatably connected to a drive shaft of the motor; and a crank arm having a proximal end pivotably connected to a distal end of the crank shaft, wherein a distal end of the crank arm is pivotably connected to a proximal end of the push rod.

A distal end of the push rod of the control assembly may be connected to the tubular syringe piston.

An axis of translation of the tubular syringe piston of the tank assembly and an axis of translation of the push rod of the control assembly may be substantially parallel to one another.

A rate of fluid flow may be regulated by a speed of the motor which is adjusted by the motor speed controller, whereby the pressure filtration of fluid entering the human cadaver body is adjusted.

According to another aspect of the present disclosure, a method for arterial embalming is provided. The method includes withdrawing an embalming fluid from a reservoir; communicating the withdrawn embalming fluid to a fluid pressure gauge; then communicating the withdrawn embalming fluid to a human cadaver body; monitoring a fluid pressure on the fluid pressure gauge which is indicative of a pressure filtration of embalming fluid entering the human cadaver body; and adjusting a rate of communication of the withdrawn embalming fluid to the human cadaver body to adjust the fluid pressure and the pressure filtration of embalming fluid entering the human cadaver body.

The rate of communication of the withdrawn embalming fluid to the human cadaver body may be decreased when the pressure filtration of fluid entering the human cadaver body is greater than a maximum threshold pressure.

The rate of communication of the withdrawn embalming fluid to the human cadaver body may be increased when the pressure filtration of fluid entering the human cadaver body is less than a minimum threshold pressure.

The communicating of the withdrawn embalming fluid to the human cadaver body may be accomplished using a motor.

The rate of communication of the withdrawn embalming fluid to the human cadaver body may include varying a speed of the motor.

The varying of the speed of the motor may be accomplished using a motor speed controller.

The withdrawing of the embalming fluid from the reservoir may be accomplished using a one-way pump assembly.

According to a further aspect of the present disclosure, a portable machine for arterial embalming is provided and includes a tank assembly, and a control assembly associated with the tank assembly.

The tank assembly includes a fluid tank defining a reservoir of fluid for embalming a human cadaver body; and a syringe vessel assembly supported on the fluid tank and extending into the reservoir of the fluid tank, wherein the syringe vessel assembly includes a fluid input and a fluid output.

The control assembly includes a fluid pressure gauge fluidly connected to the fluid output of the syringe vessel assembly; a fluid injection tube having a proximal end fluidly connected to the fluid pressure gauge and a distal end configured for injection into the human cadaver body; a motor connected to a proximal end of a push rod, a distal end of the push rob being connected to the syringe vessel assembly, wherein activation of the motor drives the push rod to actuate the syringe vessel assembly to produce a one-way flow of fluid from the reservoir of the fluid tank to the fluid pressure gauge and to the fluid injection tube; and a motor speed controller connected to the motor, wherein actuation of the motor speed controller varies a speed of the motor and varies a maximum fluid pressure measured by the fluid pressure gauge.

The fluid pressure gauge measures pressure filtration of fluid entering the human cadaver body.

The syringe vessel assembly may include an outer tubular sleeve having a distal end disposed within the reservoir of the fluid tank, wherein the outer tubular sleeve of the syringe vessel assembly includes an opening formed in a distal end thereof, wherein the distal end of the outer tubular sleeve is in close proximity to a bottom of the fluid tank; a tubular syringe piston translatably disposed within the outer tubular sleeve, wherein a proximal end of the tubular syringe piston defines the fluid output and is fluidly connected to the fluid pressure gauge, wherein the tubular syringe piston of the syringe vessel assembly includes an opening formed in a distal end thereof; a seal interposed between an outer surface of the tubular syringe piston and an inner surface of the outer tubular sleeve; and a ball disposed within the tubular syringe piston, wherein the ball has a diameter that is larger than a diameter of the opening formed in the distal end of the tubular syringe piston.

The push rod of the control assembly may be connected to the tubular syringe piston, wherein an axis of translation of the tubular syringe piston of the tank assembly and an axis of translation of the push rod of the control assembly are substantially parallel to one another.

A rate of fluid flow may be regulated by a speed of the motor which is adjusted by the motor speed controller, whereby the pressure filtration of fluid entering the human cadaver body is adjusted.

### BRIEF DESCRIPTION OF THE DRAWINGS

Various embodiments of the present disclosure are described herein below with references to the drawings, wherein:

FIG. 1 is a schematic diagram illustrating an exemplary embodiment of a speed controlled syringe pump embalming machine, in accordance with the present disclosure;

FIG. 2 is a diagram illustrating of a front view of the embalming machine coupled to a fluid tank, in accordance with the present disclosure;

FIG. 3 is a diagram illustrating a back view of the embalming machine coupled to a fluid tank, in accordance with the present disclosure;

FIG. 4 is a diagram illustrating the embalming machine, in accordance with the present disclosure, with a front panel removed from a control box housing thereof;

FIG. 5 is a diagram illustrating the embalming machine of FIG. 2, with a portion of a fluid tank broken away;

FIG. 6 is a longitudinal, cross-sectional diagram of a syringe vessel of the embalming machine of the present disclosure;

FIG. 7 is an enlarged diagram, with parts removed, illustrating components within a control assembly of the embalming machine of the present disclosure; and

FIG. 8 is a flow chart illustrating a method for arterial embalming using the embalming machine, in accordance with the present disclosure.

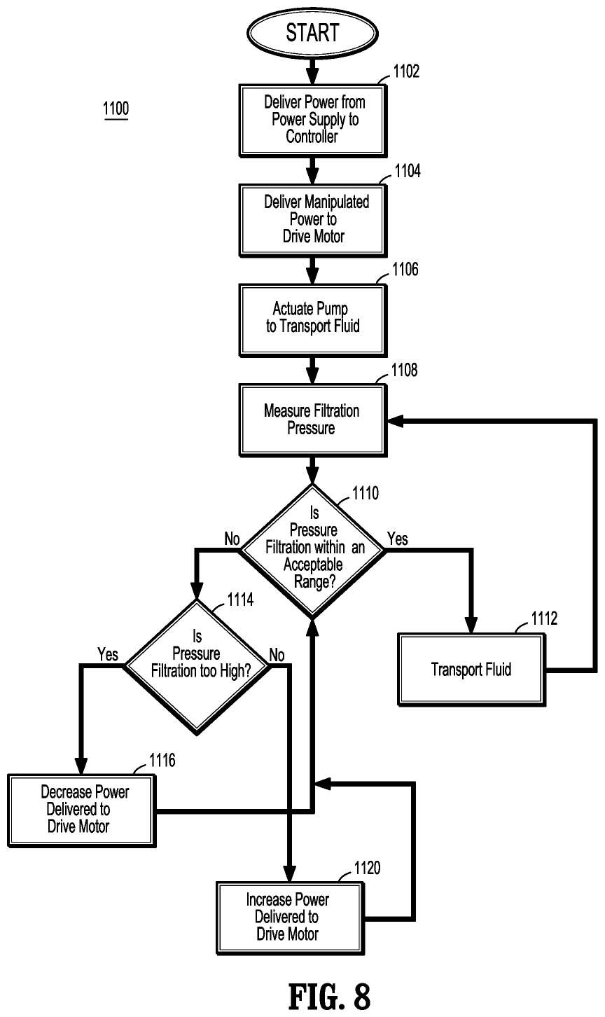

### DETAILED DESCRIPTION

The modern method of embalming involves the injection of various chemical solutions into a body's arterial network. Such chemical solutions primarily disinfect the body and slow the decomposition process. Embalming machines are used to distribute embalming fluid throughout the vascular system and tissue of the human body. When the fluid fills the arteries, vascular pressure increases throughout vascular system, which causes the fluid to move throughout the body. This process may cause portions of the body being embalmed to bulge, such bulging is unwanted for funeral viewing purposes. In existing embalming method, a drain tube or drainage forceps may be used, and an embalmer may open the drain tube periodically to allow blood to escape, relieve the vascular pressure, and counter undesired bulging.

In accordance with the present disclosure, a portable speed controlled embalming machine is presented as illustrated in the attached figures. The speed controlled machine may include four integral components, namely a drive motor, a motor speed controller, a syringe vessel, and a pressure gauge. The motor speed controller may be configured to control the power delivered to the drive motor. In one embodiment, the motor speed controller may be electrically powered, or powered by any other suitable means. The syringe vessel may be capable of drawing and releasing fluid by linear motion or any other suitable motion. The pressure gauge may read the positive pressure created within the human body during embalming which results in movement of the embalming fluid from the arterial system to tissue, and then to the venous system. This positive pressure which is created is called pressure filtration. Additional components may include, but are not limited to: ball valves, needle valves, and/or fluid tanks.

In accordance with the present disclosure, the drive motor may actuate a pump to transport fluid(s) to and from a fluid tank and a human body. The drive motor, by rotation, may motion a push rod by linear actuation, or by any other suitable means. By linear actuation, the push rod, which also may be connected to a syringe vessel, may control the drawing and expelling of fluid to and from a fluid tank. The fluid may be displaced throughout a human body for arterial embalming via the syringe vessel. The tubing may have a combination of all or some of the components described herein above or already known and used by those skilled in the art. These components may give an operator additional control over the properties of the fluid displaced, and more control over the pressure filtration.

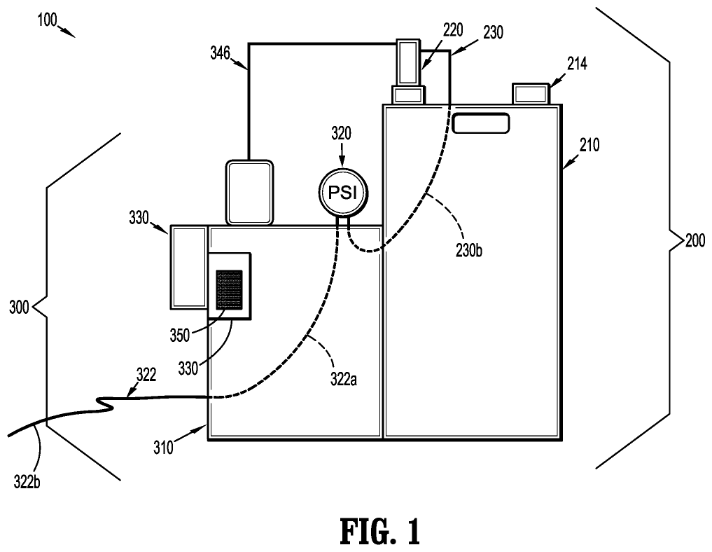

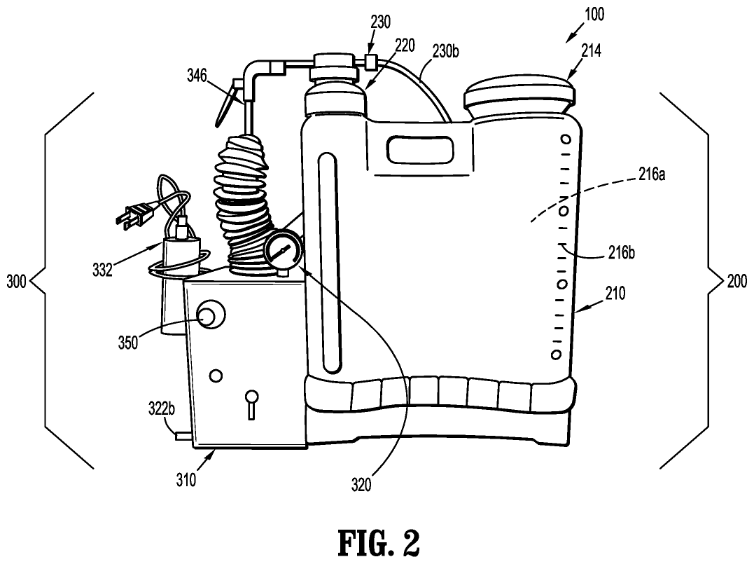

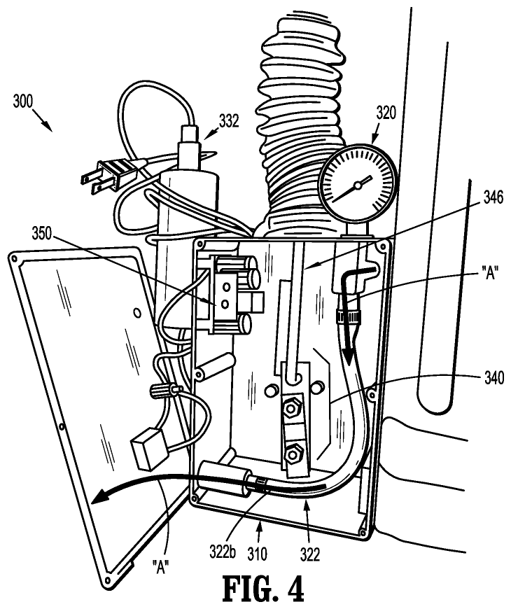

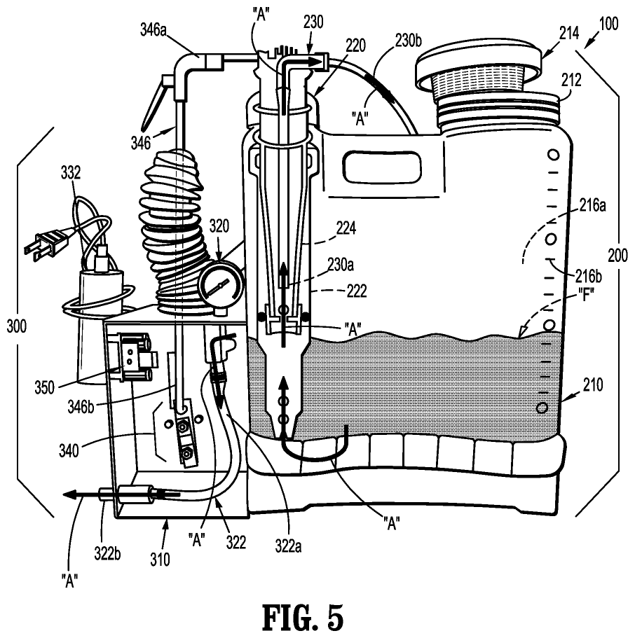

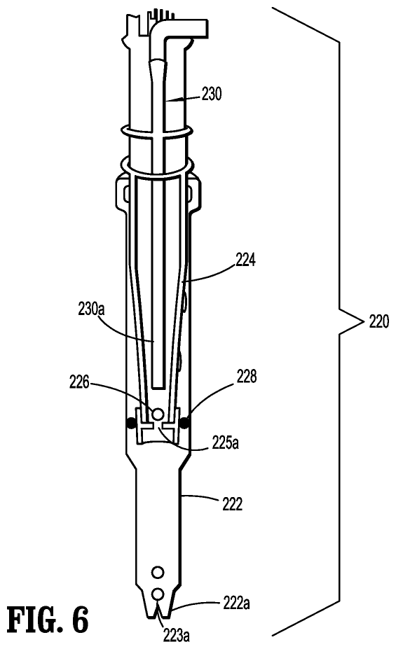

With reference to FIGS. 1-6, a speed controlled syringe pump embalming machine 100 is shown. Embalming machine 100 includes a tank assembly 200, and a control assembly 300.

Tank assembly 200 includes a fluid tank 210 defining a closable and sealable opening 212 via cap 214. Fluid tank 210 defines a cavity or reservoir 216a therein. Fluid tank 210 includes graduation marks 216b thereon for providing an indication of a level of fluid “F” within reservoir 216a.

Tank assembly 200 further includes a syringe vessel assembly 220 supported on fluid tank 210 and extending into reservoir 216a of fluid tank 210. Syringe vessel assembly 220 is configured to function as a one-way fluid valve to draw fluid from within reservoir 216a of fluid tank 210 and deliver the fluid to control assembly 300 and into a human body (e.g., cadaver).

Syringe vessel assembly 220 includes an outer tubular sleeve 222 having a distal end 222a disposed within reservoir 216 of fluid tank 210 and extending an amount sufficient to be in close proximity to a bottom of fluid tank 210. The distal end 222a of outer tubular sleeve 222 defines an opening or bore 223a therein. Syringe vessel assembly 220 includes a tubular syringe piston or plunger 224 translatably disposed within the outer tubular sleeve 222. Tubular syringe plunger 224 defines an opening 225a in a distal end 224a thereof defining a fluid input. A ball 226 is disposed within tubular syringe plunger 224 and is dimensioned to close or seal opening 225a of tubular syringe plunger 224 during an up stroke of tubular syringe plunger 224 relative to outer tubular sleeve 222, and open the opening 225a of tubular syringe plunger 224 during a down stroke of tubular syringe plunger 224 relative to outer tubular sleeve 222. A bushing, o-ring, seal or the like 228 is provided between an inner surface of outer tubular sleeve 222 and an outer surface of tubular syringe plunger 224 to maintain a fluid tight seal therebetween.

Syringe vessel assembly 220 includes a fluid output tube 230 having a distal end 230a fluidly disposed within tubular syringe plunger 224, or otherwise fluidly connected to tubular syringe plunger 224. Fluid output tube 230 includes a proximal end 230b connected to a fluid output of the syringe vessel assembly 220 and extending to control assembly 300.

Syringe vessel assembly 220 may, but is not limited to, displace approximately 2 oz of fluid per stroke.

Control assembly 300 includes a control housing, box or cabinet 310 connected to or supporting tank assembly 200. Control assembly 300 further includes a fluid pressure gauge 320 supported on control housing 310. Fluid pressure gauge 320 is in fluid communication with proximal end 230b of fluid output tube 230 of tank assembly 200.

Control assembly 300 also includes a fluid injection tube 322 having a proximal end 322a in fluid communication with pressure gauge 320, and a distal end 322b configured for injection into a human body (e.g., cadaver), or configured to support a needle (not shown) which is configured for injection into a human body (e.g., cadaver).

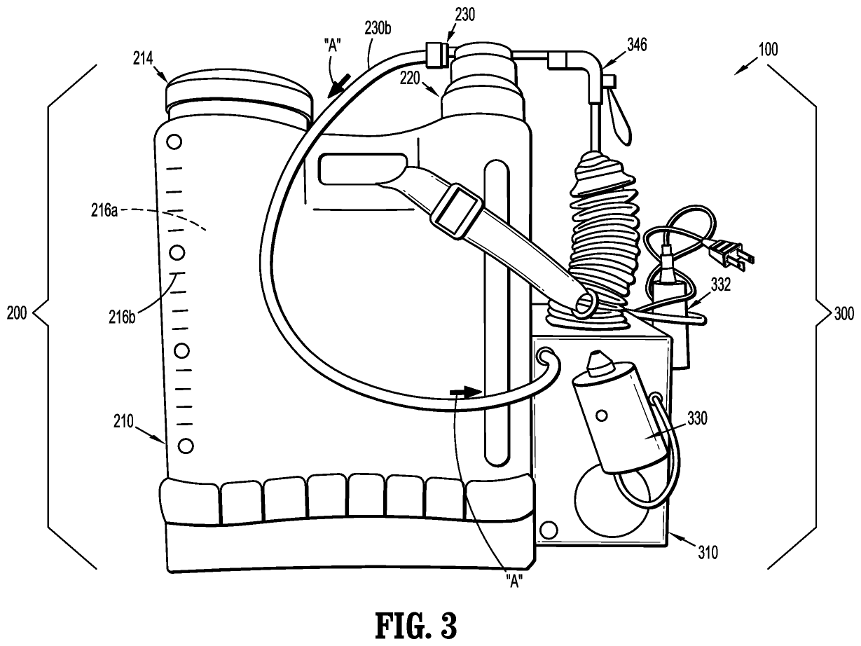

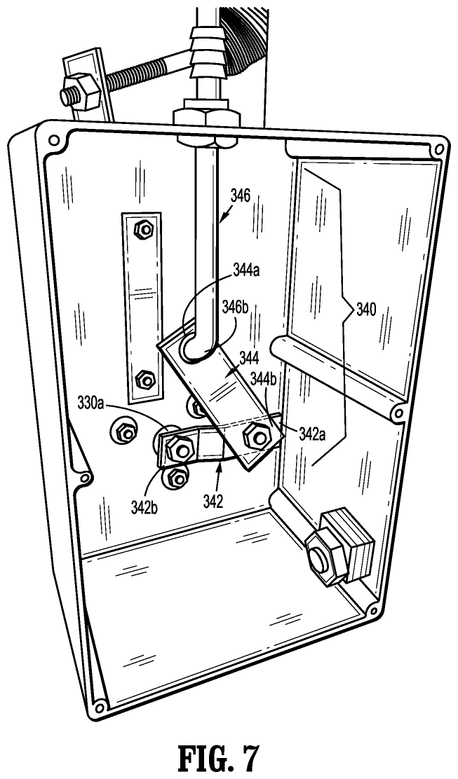

Control assembly 300 further includes a motor 330 supported on or in control housing 310. Motor 330 may be driven by connection to a power source (e.g., electrical outlet) via an electrical plug 332, or may be driven by a portable power source, such as, for example, a battery, hand crank, etc. With specific reference to FIGS. 3 and 7, motor 330 includes a drive shaft 330a mechanically connected to a drive assembly 340. Specifically, drive shaft 330a of motor 330 non-rotatably supports a proximal end 342b of crank shaft 342 of drive assembly 340. A distal end 342a of crank shaft 342 is pivotably connected to a proximal end 344b of a crank arm 344 of drive assembly 340. A distal end 344a of crank arm 344 is pivotably connected to a proximal end 346b of a push rod 346 of drive assembly 340. A distal end 346a of push rod 346 is connected to fluid output tube 230 of syringe vessel assembly 220 of tank assembly 200. Push rod 346 is supported in control housing 310 in such a manner that axial translation thereof is parallel to axial translation of tubular syringe plunger 224 of syringe vessel 220 of tank assembly 200.

In operation, as motor 330 rotates drive shaft 330a, drive shaft 330 rotates crank shaft 342, which in turn exerts a force on crank arm 344. Crank arm 344 in turn exerts a force on push rod 346 to cause push rod 346 to axially translate along a longitudinal axis thereof. Since distal end 346a of push rod 346 is connected to tubular syringe plunger 224 of syringe vessel 220, push rod 346 causes tubular syringe plunger 224 to axially translate relative to outer tubular sleeve 222 of syringe vessel 220 of tank assembly 200.

As such, during an up stroke of tubular syringe plunger 224 relative to outer tubular sleeve 222, since ball 226 of syringe vessel 220 is held in a closed condition against seal opening 225a of tubular syringe plunger 224, fluid “F” is drawn into outer tubular sleeve 222 of syringe vessel 220 from within reservoir 216a of fluid tank 210. Further, during a down stroke of tubular syringe plunger 224 relative to outer tubular sleeve 222, since ball 226 of syringe vessel 220 now opens seal opening 225a of tubular syringe plunger 224, fluid “F” is pushed into fluid output tube 230 of syringe vessel assembly 220, into or across pressure gauge 320 of control assembly 300, and ultimately into the human body (e.g., cadaver), as indicated by arrows “A”.

As fluid “F” is being pumped into the human body (e.g., cadaver), pressure gauge 320 of control assembly 300 is measuring vascular resistance or pressure filtration of the fluid “F” as it enters the human body (e.g., cadaver), due to the density of the tissue, rate of passage of fluid from the arterial system to the venous system (through the tissue of the human body or cadaver).

Motor 330 may be sized and configured to complete approximately 60 strokes per minute.

In accordance with the present disclosure, control assembly 300 may include a motor speed controller 350 operatively associated with motor 330 which functions to vary the speed of motor 330. In operation, as motor 330 is driving push rod 346 to pump fluid “F” into the human body (e.g., cadaver), the vascular resistance or pressure filtration is being measure by pressure gauge 320 of control assembly 300, and monitored by the operator (e.g., embalmer). If a maximum measured pressure, as measured by pressure gauge 320, is greater than a desired pressure, the operator my manipulate motor speed controller 350 to slow down the speed of motor 330, and in turn, reduce the maximum measured pressure. Similarly, if a maximum measured pressure, as measured by pressure gauge 320, is less than a desired pressure, the operator my manipulate motor speed controller 350 to speed up the speed of motor 330, and in turn, increase the maximum measured pressure.

It is contemplated that control system 300 may include a control board or processor (not shown) connected to motor 330, motor speed controller 350 and pressure gauge 320, whereby the control board can be preprogrammed to a predetermined maximum pressure level and continuously monitor pressure gauge 320 during operation of motor 330, and automatically adjust the speed of motor 330, via motor speed controller 350, to maintain the measured pressure within a desired range of pressures. In accordance with the present disclosure, and more specifically, it is further contemplated that motor speed controller 350 may operate remotely and/or automatically (e.g., without user interaction during the embalming procedure), wherein the user may input a desired maximum and/or minimum pressure level (e.g., pressure filtration) for pressure gauge 320, whereby the motor speed controller 350 automatically adjusts the speed of motor 330 during the embalming procedure so as to remain between the maximum and/or minimum pressure level (e.g., pressure filtration), whereby the user/embalmer/operator can remain focused on the human body or cadaver without stepping away therefrom to observer/monitor pressure gauge 320 and/or make manual adjustments to the motor speed controller 350 of embalming machine 100.

With reference to FIG. 11, a flowchart illustrating a method for arterial embalming using the speed controlled syringe pump embalming machine 100 is shown in accordance with the present disclosure. At step 1102, electrical power may be delivered from power supply 332 to motor speed controller 350. At step 1104, electrical power may be delivered to drive motor 330 after the power has been manipulated by motor speed controller 350. Motor speed controller 350 may increase or decrease the voltage that is delivered from power supply 332 to drive motor 330, thereby increasing or decreasing the electrical power that is delivered to drive motor 330. At step 1106, drive motor 330 may actuate a one way pump (e.g., syringe vessel assembly 220) to facilitate the transportation of fluid(s) “F” from fluid tank 210 to a human body or cadaver, via fluid output tube 230.

At step 1108, the pressure filtration is measured by pressure gauge 320. At step 1110, a controller (not shown) or the operator will determine whether the pressure filtration is within an acceptable range. If the pressure filtration is within an acceptable range, then at step 1112, fluid(s) “F” may continue to be transported from fluid tank 210 to the human body or cadaver. If the pressure filtration is determined to be outside of an acceptable range (e.g., the pressure filtration is too high), then at step 1114, the motor speed controller 350 may be manipulated to decrease the power delivered to drive motor 330. If the pressure filtration is not too high, then the motor speed controller 350 may be manipulated to increase the power delivered to the drive motor 330.

The speed of drive motor 330 may be automatically regulated by the motor speed controller 350 so that pressure filtration is constantly within an acceptable range while fluid(s) “F” is/are transported to and from a fluid tank and a human body or cadaver. By using a motor speed controller 350, manual discharge of fluids (e.g., blood, bodily fluids, embalming fluid, etc.) may be eliminated. Also, by manipulating the electrical power delivered to drive motor 330, the flow of fluid “F” may be regulated. By regulating the flow of fluid “F” into a human body or cadaver, pressure filtration may be regulated and maintained within an acceptable predetermined range. By maintaining the pressure filtration within an acceptable range, the operator may prevent unwanted consequences during the embalming process (e.g., bulging of veins, popping of eyes, etc.).

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A portable machine for arterial embalming, the embalming machine comprising:
a tank assembly, including:
a fluid tank defining a reservoir of fluid for embalming a human cadaver body; and
a syringe vessel assembly supported on the fluid tank and extending into the reservoir of the fluid tank, wherein the syringe vessel assembly is a one-way fluid valve, and wherein the syringe vessel assembly includes a fluid input and a fluid output; and
a control assembly associated with the tank assembly, the control assembly including:
a fluid pressure gauge fluidly connected to the fluid output of the syringe vessel assembly;
a fluid injection tube having a proximal end fluidly connected to the fluid pressure gauge and a distal end configured for injection into the human cadaver body;
a motor connected to a proximal end of a push rod, a distal end of the push rob being connected to the syringe vessel assembly, wherein activation of the motor drives the push rod to actuate the syringe vessel assembly to produce a one-way flow of fluid from the reservoir of the fluid tank to the fluid pressure gauge and to the fluid injection tube; and
a motor speed controller connected to the motor, wherein actuation of the motor speed controller varies a speed of the motor and varies a maximum fluid pressure measured by the fluid pressure gauge,
wherein the fluid pressure gauge measures pressure filtration of fluid entering the human cadaver body.

2. The embalming machine of claim 1, wherein the syringe vessel assembly includes:
an outer tubular sleeve having a distal end disposed within the reservoir of the fluid tank;
a tubular syringe piston translatably disposed within the outer tubular sleeve, wherein a proximal end of the tubular syringe piston defines the fluid output and is fluidly connected to the fluid pressure gauge; and
a seal interposed between an outer surface of the tubular syringe piston and an inner surface of the outer tubular sleeve.

3. The embalming machine of claim 2, wherein the outer tubular sleeve of the syringe vessel assembly includes an opening formed in a distal end thereof, wherein the distal end of the outer tubular sleeve is in close proximity to a bottom of the fluid tank.

4. The embalming machine of claim 3, wherein the tubular syringe piston of the syringe vessel assembly includes an opening formed in a distal end thereof, and wherein the syringe vessel assembly includes a ball disposed within the tubular syringe piston, wherein the ball has a diameter that is larger than a diameter of the opening formed in the distal end of the tubular syringe piston.

5. The embalming machine of claim 4, wherein the push rod of the control assembly is connected to the tubular syringe piston.

6. The embalming machine of claim 4, wherein the control assembly includes:
a crank shaft having a proximal end non-rotatably connected to a drive shaft of the motor; and
a crank arm having a proximal end pivotably connected to a distal end of the crank shaft, wherein a distal end of the crank arm is pivotably connected to a proximal end of the push rod.

7. The embalming machine of claim 6, wherein a distal end of the push rod of the control assembly is connected to the tubular syringe piston.

8. The embalming machine of claim 7, wherein an axis of translation of the tubular syringe piston of the tank assembly and an axis of translation of the push rod of the control assembly are substantially parallel to one another.

9. The embalming machine of claim 8, wherein a rate of fluid flow is regulated by a speed of the motor which is adjusted by the motor speed controller, whereby the pressure filtration of fluid entering the human cadaver body is adjusted.

10. A method for arterial embalming, the method comprising:
withdrawing an embalming fluid from a reservoir;
communicating the withdrawn embalming fluid to a fluid pressure gauge;
then communicating the withdrawn embalming fluid to a human cadaver body;
monitoring a fluid pressure on the fluid pressure gauge which is indicative of a pressure filtration of embalming fluid entering the human cadaver body; and
adjusting a rate of communication of the withdrawn embalming fluid to the human cadaver body to adjust the fluid pressure and the pressure filtration of embalming fluid entering the human cadaver body.

11. The method of claim 10, wherein the rate of communication of the withdrawn embalming fluid to the human cadaver body is decreased when the pressure filtration of fluid entering the human cadaver body is greater than a maximum threshold pressure.

12. The method of claim 10, wherein the rate of communication of the withdrawn embalming fluid to the human cadaver body is increased when the pressure filtration of fluid entering the human cadaver body is less than a minimum threshold pressure.

13. The method of claim 11, wherein the communicating of the withdrawn embalming fluid to the human cadaver body is accomplished using a motor.

14. The method of claim 13, wherein the adjusting of the rate of communication of the withdrawn embalming fluid to the human cadaver body includes varying a speed of the motor.

15. The method of claim 14, wherein the varying of the speed of the motor is accomplished using a motor speed controller.

16. The method of claim 15, wherein the withdrawing of the embalming fluid from the reservoir is accomplished using a one-way pump assembly.

17. A portable machine for arterial embalming, the embalming machine comprising:
a tank assembly, including:
a fluid tank defining a reservoir of fluid for embalming a human cadaver body; and
a syringe vessel assembly supported on the fluid tank and extending into the reservoir of the fluid tank, wherein the syringe vessel assembly includes a fluid input and a fluid output; and
a control assembly associated with the tank assembly, the control assembly including:
a fluid pressure gauge fluidly connected to the fluid output of the syringe vessel assembly;
a fluid injection tube having a proximal end fluidly connected to the fluid pressure gauge and a distal end configured for injection into the human cadaver body;
a motor connected to a proximal end of a push rod, a distal end of the push rob being connected to the syringe vessel assembly, wherein activation of the motor drives the push rod to actuate the syringe vessel assembly to produce a one-way flow of fluid from the reservoir of the fluid tank to the fluid pressure gauge and to the fluid injection tube; and
a motor speed controller connected to the motor, wherein actuation of the motor speed controller varies a speed of the motor and varies a maximum fluid pressure measured by the fluid pressure gauge,
wherein the fluid pressure gauge measures pressure filtration of fluid entering the human cadaver body.

18. The embalming machine of claim 17, wherein the syringe vessel assembly includes:
an outer tubular sleeve having a distal end disposed within the reservoir of the fluid tank, wherein the outer tubular sleeve of the syringe vessel assembly includes an opening formed in a distal end thereof, wherein the distal end of the outer tubular sleeve is in close proximity to a bottom of the fluid tank;
a tubular syringe piston translatably disposed within the outer tubular sleeve, wherein a proximal end of the tubular syringe piston defines the fluid output and is fluidly connected to the fluid pressure gauge, wherein the tubular syringe piston of the syringe vessel assembly includes an opening formed in a distal end thereof;
a seal interposed between an outer surface of the tubular syringe piston and an inner surface of the outer tubular sleeve; and
a ball disposed within the tubular syringe piston, wherein the ball has a diameter that is larger than a diameter of the opening formed in the distal end of the tubular syringe piston.

19. The embalming machine of claim 18, wherein the push rod of the control assembly is connected to the tubular syringe piston, wherein an axis of translation of the tubular syringe piston of the tank assembly and an axis of translation of the push rod of the control assembly are substantially parallel to one another.

20. The embalming machine of claim 19, wherein a rate of fluid flow is regulated by a speed of the motor which is adjusted by the motor speed controller, whereby the pressure filtration of fluid entering the human cadaver body is adjusted.
